(19) United States
(12) Patent Application Publication
(10) Pub. No.: US 2009/0024084 A1
(43) Pub. Date: Jan. 22, 2009

(54) MULTI-LUMEN CATHETER ASSEMBLY AND METHOD OF PROVIDING RELATIVE MOTION THERETO

(75) Inventors: Rajesh K. Khosla, Beachwood, OH (US); Steve Christian, New Brighton, MN (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: PeriTec Biosciences Ltd.

(21) Appl. No.: 12/173,603

(22) Filed: Jul. 15, 2008

Related U.S. Application Data
(60) Provisional application No. 60/959,736, filed on Jul. 16, 2007.

(51) Int. Cl. A61M 25/01

(52) U.S. Cl. 604/95.01

(57) ABSTRACT

A handle for providing relative motion to a multi-lumen catheter assembly includes an elongated handle body having a longitudinal axis. The handle body has longitudinally spaced proximal and distal handle ends. An outer catheter chuck is connected to the distal handle end and is adapted to receive an outer catheter member. An inner catheter chuck is connected to the proximal handle end and is adapted to receive an inner catheter member. A handle actuator is movably connected to the handle body. The handle actuator is adapted to receive an intermediate catheter member and to move the intermediate catheter member relative to the inner and outer catheter members. The intermediate catheter is at least partially interposed between the inner and outer catheter.

### RELATED APPLICATION

This application claims the benefit of U.S. Provisional Patent Application Ser. No. 60/959,736, filed Jul. 16, 2007 which is incorporated herein by reference.

### TECHNICAL FIELD

The present invention relates to an apparatus and method for use of a multi-lumen catheter assembly and, more particularly, to a handle for providing relative motion to a multi-lumen catheter assembly and a method of using the handle.

### BACKGROUND OF THE INVENTION

In many minimally invasive surgical procedures, such as percutaneous procedures, a catheter is inserted into a patient's body to facilitate remote manipulation of surgical tools at a surgery site within the patient. For example, a user may desire to deploy an intraluminal prosthesis within a patient's vasculature, possibly with the assistance of an apparatus such as that disclosed in the commonly assigned U.S. patent application Ser. No. 11/601,075, filed Nov. 16, 2006 for APPARATUS AND METHOD FOR DELIVERING LINED INTRALUMINAL PROSTHESES. However, the user must have some way of controlling the deployment of the intraluminal prosthesis deep within the body.

Often such deployment control or other remote control during a percutaneous procedure is provided by relative motion of various structures of a catheter assembly. However, it can be difficult for a user to move multiple structures in different directions, possibly at different rates of motion, while monitoring conditions at the surgery site through remote viewing devices.

### SUMMARY OF THE INVENTION

In an embodiment of the present invention, a handle for providing relative motion to a multi-lumen catheter assembly is described. An elongated handle body has a longitudinal axis. The handle body has longitudinally spaced proximal and distal handle ends. An outer catheter chuck is connected to the distal handle end and is adapted to receive an outer catheter member. An inner catheter chuck is connected to the proximal handle end and is adapted to receive an inner catheter member. A handle actuator is movably connected to the handle body. The handle actuator is adapted to receive an intermediate catheter member and to move the intermediate catheter member relative to the inner and outer catheter members. The intermediate catheter is at least partially interposed between the inner and outer catheter.

In an embodiment of the present invention, a multi-lumen catheter assembly is described. An outer catheter member has a longitudinal axis and longitudinally spaced proximal and distal outer catheter ends with a first lumen defined therebetween. An intermediate catheter member has longitudinally spaced proximal and distal intermediate catheter ends with a second lumen defined therebetween. The intermediate catheter member is at least partially located within the first lumen. An inner catheter member has longitudinally spaced proximal and distal inner catheter ends. The inner catheter member is at least partially located within the second lumen. A handle is connected to the proximal outer catheter end, the proximal intermediate catheter end, and the proximal inner catheter end. The handle is adapted to maintain the relative position of the outer and inner catheter members while providing movement of the intermediate catheter member relative thereto.

In an embodiment of the present invention, a method of providing relative motion to a multi-lumen catheter assembly is described. An outer catheter member having a longitudinal axis and longitudinally spaced proximal and distal outer catheter ends with a first lumen defined therebetween is provided. An intermediate catheter member is at least partially located within the first lumen. The intermediate catheter member has longitudinally spaced proximal and distal intermediate catheter ends with a second lumen defined therebetween. An inner catheter member is at least partially located within the second lumen. The inner catheter member has longitudinally spaced proximal and distal inner catheter ends. A handle is connected to the proximal outer catheter end, the proximal intermediate catheter end, and the proximal inner catheter end. The relative position of the outer and inner catheter members is maintained. Movement of the intermediate catheter member relative to the outer and inner catheter members is provided.

### BRIEF DESCRIPTION OF THE DRAWINGS

For a better understanding of the invention, reference may be made to the accompanying drawings, in which:

FIG. 1 is a schematic side view of one embodiment of the present invention;

FIG. 2 is a cross-sectional view taken along line 2-2 of FIG. 1;

FIG. 3 is a partial side view of the embodiment of FIG. 1;

FIG. 4 is a cross-sectional view taken along line 4-4 of FIG. 3;

FIG. 5 is a magnified view of area 5 of FIG. 4;

FIG. 6A is a perspective view of the embodiment of FIG. 1 in a first condition;

FIG. 6B is a perspective view of the embodiment of FIG. 1 in a second condition; and

FIG. 6C is a perspective view of the embodiment of FIG. 1 in a third condition.

### DESCRIPTION OF EMBODIMENTS

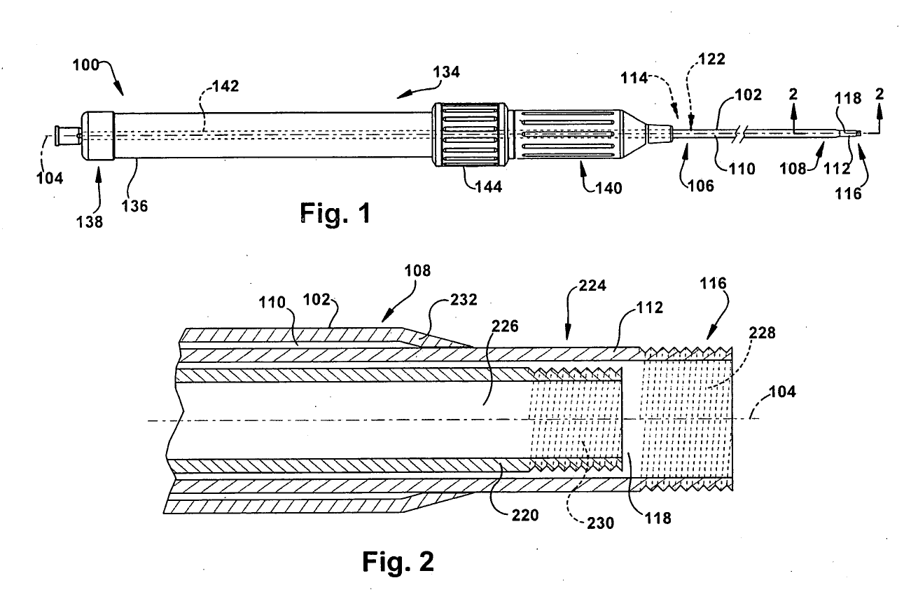

In accordance with the present invention, FIG. 1 depicts a schematic side view of a multi-lumen catheter assembly 100. An outer catheter member 102 has a longitudinal axis 104 and longitudinally spaced proximal and distal outer catheter ends 106 and 108, respectively, with a first lumen 110 defined therebetween. The outer catheter member 102 may be dimensioned as desired for use in a particular application of the present invention. Like many longitudinally oriented structures described herein, only a portion of the length of the outer catheter member 102 is shown, for ease of depiction.

An intermediate catheter member 112 is at least partially located within the first lumen 110. The intermediate catheter member has longitudinally spaced proximal and distal intermediate catheter ends 114 (hidden within the outer catheter member 102 in this view) and 116, respectively, with a second lumen 118 defined therebetween.

An inner catheter member 220 is at least partially located within the second lumen 118. The inner catheter member 220 is not visible in FIG. 1. FIG. 2, however, is a cross-sectional view taken along line 2-2 of FIG. 1 and depicts the inner catheter member 220, which has longitudinally spaced proximal and distal inner catheter ends 122 (not shown in FIG. 2, hidden in FIG. 1) and 224, respectively. A third lumen 226 may be defined between the proximal and distal inner catheter ends 122 and 224.

As depicted in FIG. 2, any combination of the outer, intermediate, and inner catheter members 102, 112, and 220 may be at least partially arranged in a mutually coaxial relationship (shown here as being coaxial about the longitudinal axis 104). A mutually coaxial relationship, when one exists between two or more of the outer, intermediate, and inner catheter members 102, 112, and 220, need not extend along the entire length of the “mutually coaxial” members. The outer, intermediate, and inner catheter members 102, 112, and 220 may each define a separate axis (not shown), which may have any desired collinear, parallel, other, or no relationship with a corresponding axis (not shown) of another of the outer, intermediate, and inner catheter members, either in whole or in part of the length of such axes.

One or more of the distal outer catheter end 108, the distal intermediate catheter end 116, and the distal inner catheter end 224 may be adapted for operative connection to a intraluminal prosthesis cartridge (not shown) as part of a intraluminal prosthesis delivery system (not shown). To facilitate such connection, the chosen one(s) of the distal outer catheter end 108, the distal intermediate catheter end 116, and the distal inner catheter end 224 may include a coupling or fitting member of any suitable type, such as the intermediate catheter threaded connector 228 or the inner catheter threaded connector 230 shown in FIG. 2. Additionally or alternatively, one or more of the distal outer catheter end 108, the distal intermediate catheter end 116, and the distal inner catheter end 224 may include a distal end profile shape, such as the outer catheter beveled portion 232 of FIG. 2. When present, a shaped profile may assist in streamlining and smoothing the interface between adjacent catheter members 102, 112, and/or 220 and helping avoid trauma to the patient's internal structures during use of the multi-lumen catheter assembly 100.

Returning to FIG. 1, the multi-lumen catheter assembly 100 also includes a handle 134 connected to at least one of the proximal outer catheter end 106, the proximal intermediate catheter end 114, and the proximal inner catheter end 122. For most applications of the present invention, the handle 134 will be connected to all of these catheter ends 106, 113, and 122, and the following discussion will presume such. However, one of ordinary skill in the art could readily design a multi-lumen catheter assembly 100 in which at least one of the outer catheter member 102, the intermediate catheter member 112, and the inner catheter member 220 either has a non-proximal portion connected to the handle 134 or is not connected to the handle at all.

The handle 134 is adapted to maintain the relative positioning of two or more of the outer, intermediate, and inner catheter members 102, 112, and 220 while moving the remaining one(s) of these catheter members translationally and/or rotationally relative to the maintained catheter members 102, 112, and/or 220. For clarity of discussion inn the below description, the handle 134 will be presumed to be adapted to maintain the relative position of the outer and inner catheter members 102 and 220 while providing translational movement of the intermediate catheter member 112 relative thereto.

As shown schematically in FIG. 2, the handle 134 includes an elongated handle body 136 having longitudinally spaced proximal and distal handle ends 138 and 140, respectively, with the proximal handle end 138 being affixed to the proximal inner catheter end 122 in any suitable manner, as will be discussed shortly. A handle lumen 142 extends longitudinally between the proximal and distal handle ends 138 and 140. The inner catheter 122, being affixed to the proximal handle end 138, extends longitudinally through the handle lumen 142.

A handle actuator 144 is affixed to the proximal intermediate catheter end 114 and is movably connected to the handle body 136. The handle actuator 144 may be movable in as many degrees of freedom as desired, and is adapted for both selective axial rotation and selective longitudinal translation relative to the handle body 136 in the depicted embodiment of the present invention. The handle actuator 144 is selectively movable relative to the handle body 136 to move the attached intermediate catheter member 112 longitudinally relative to the outer and inner catheter members 102 and 220.

The handle actuator 144 may have any suitable mechanical connection with the handle body 136. For example, bearings associated with either or both of the handle actuator 144 and the handle body 136 could provide a rolling interface between the two. As another example, the handle actuator 144 may simply be slidably connected to the handle body 136. The relative materials, dimensions, and other properties of both the handle actuator 144 and the handle body 136 may be chosen to optimize frictional forces therebetween for a desired mechanical result. For example, when there is a relatively tight fit between the handle actuator 144 and the handle body 136, a resulting frictional engagement may help to retain the handle actuator in a desired position along the handle body, while a looser fit may be preferable when ease of motion is more desirable than frictional retention.

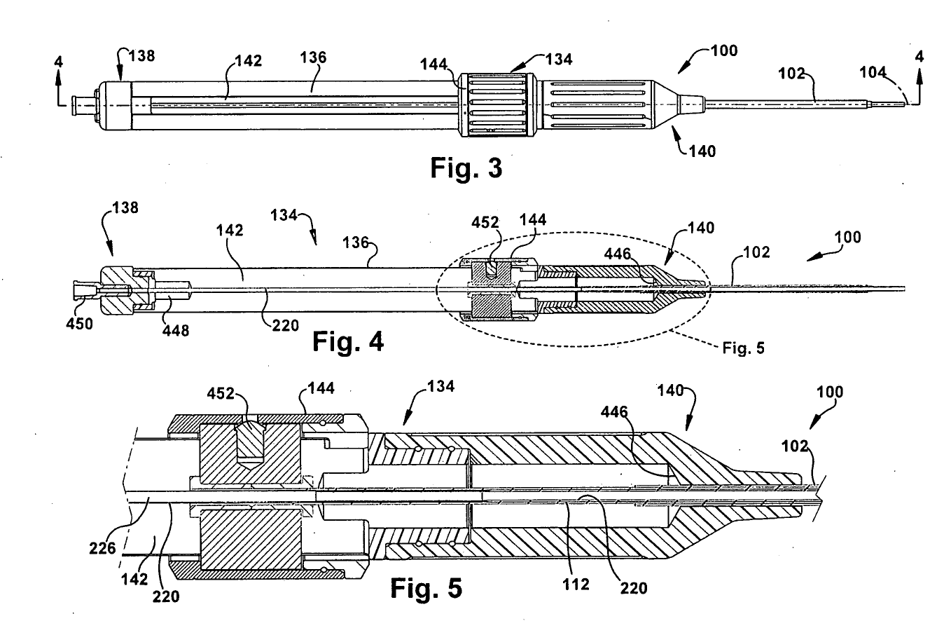

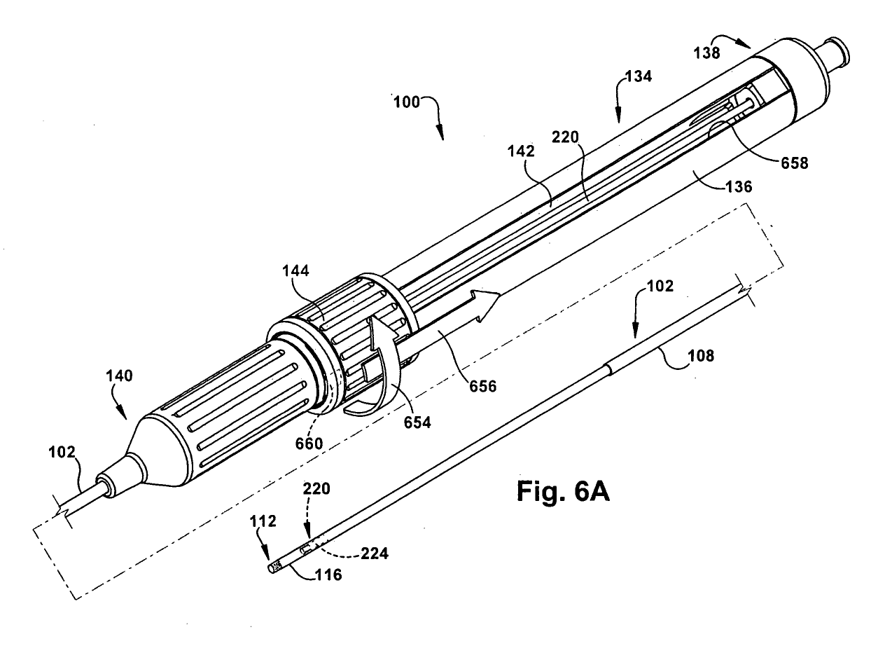

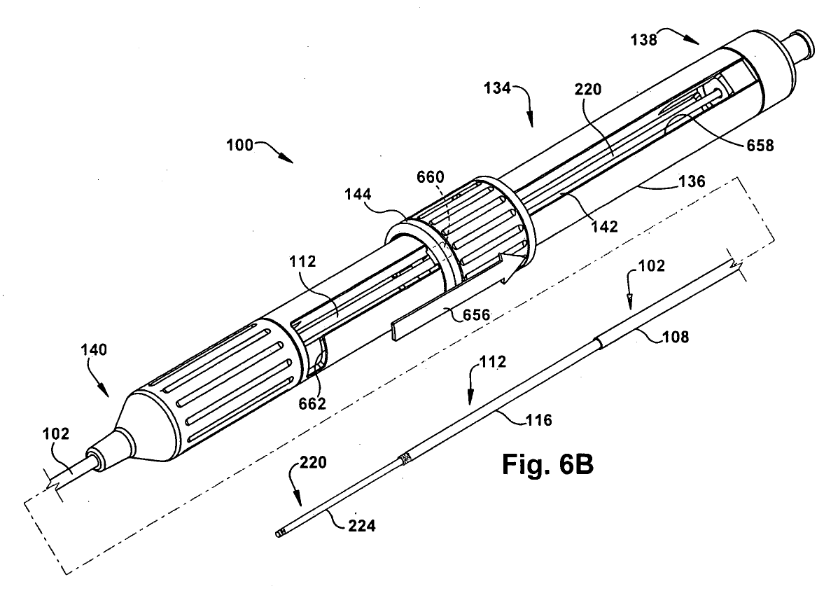

FIGS. 3-5 show the structure of the handle 134 in detail. FIG. 3 depicts a partial side view of the multi-lumen catheter assembly 100. FIG. 4 is a cross-sectional view of the multi-lumen catheter assembly 100 taken along line 4-4 in FIG. 3. As can be seen in FIG. 4, and in the magnified view of FIG. 5, an outer catheter chuck 446 is connected to the distal handle end 140 and is adapted to receive the outer catheter member 102. Similarly, an inner catheter chuck 448 is connected to the proximal handle end 138 and is adapted to receive the inner catheter member 220. The outer and inner catheter chucks 446 and 448 hold the outer and inner catheter members 102 and 220, respectively, in a stationary position with respect to the handle body 136.

Because the inner catheter chuck 448 is connected to the proximal handle end 138 and to the inner catheter member 220, the inner catheter member extends longitudinally through at least a portion of the handle body 136 when received by the inner catheter chuck. For example, the inner catheter member 220 could extend through at least a portion of the handle lumen 142, as depicted in FIG. 4.

When the inner catheter member 220 includes the optional third lumen 226, the proximal handle end 138 may include an inner catheter adapter 450, as shown in FIG. 4, placing the proximal handle end in fluid communication with the third lumen. The inner catheter adapter 450 may assist a user in providing one or more of surgical tools, prostheses, fluids, or vacuum to the surgical site through the third lumen 226. The inner catheter adapter 450 may be formed integrally with the handle body 136 or provided separately for mounting on the handle body 136, possibly as a portion/feature of the inner catheter chuck 448.

The handle actuator 144 is adapted to receive the intermediate catheter member 112 and to move the intermediate catheter member relative to the outer and inner catheter members 102 and 220. As a consequence, the handle actuator 144 is adapted to move the distal intermediate catheter end 116 longitudinally relative to the distal outer and inner catheter ends 108 and 224 and thereby to place the distal intermediate catheter end into a desired position at the surgery side within the patient's body while maintaining the position of the distal outer and inner catheter ends relative to the distal intermediate catheter end.

For example, the multi-lumen catheter assembly 100 may be arranged so that the handle actuator 144 is selectively movable relative to the handle body 136 to move the distal intermediate catheter end 116 longitudinally between the distal inner catheter end 224 and the distal outer catheter end 108. Alternatively or additionally, the handle actuator 144 may be selectively movable relative to the handle body 136 to extend the distal intermediate catheter end 116 longitudinally beyond the distal inner catheter end 224. One of ordinary skill in the art can readily choose the relative sizes of any or all structures of the multi-lumen catheter assembly 100 in order to achieve relative movement between any or all of the outer, intermediate, and inner catheter members 102, 112, and 220 as desired for a particular application of the present invention. For example, the lengths of the outer, intermediate, and inner catheter members 102, 112, and 220 may be chosen to provide desired arrangements and spacing between two or more of the distal outer, intermediate, and inner catheter ends 108, 116, and 224 during operation of the multi-lumen catheter assembly.

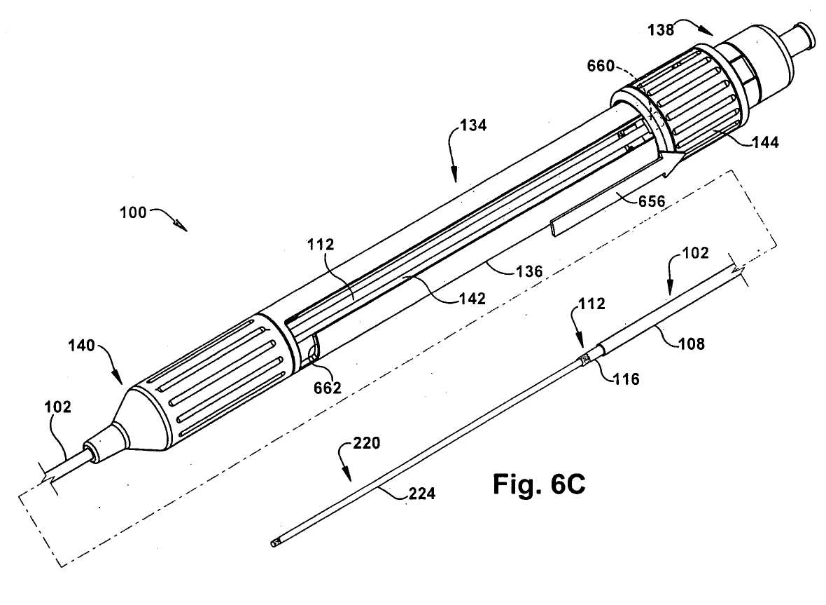

An actuator detent mechanism 452, such as the spring-loaded pin shown in FIGS. 3-5, may be provided to selectively resist motion of the handle actuator 144 with respect to the handle body 136. The actuator detent mechanism 452 may be adapted to selectively resist motion of the handle actuator 144 in a longitudinal translation direction, as described above, and/or in an axial rotation direction, as will be described below with reference to FIG. 6A. The actuator detent mechanism 452 may be readily designed for a particular application of the multi-lumen catheter assembly 100 by one of ordinary skill in the art, and may use any suitable mechanism to resist motion in the desired manner.

For example, the actuator detent mechanism 452 may be designed to lightly hold the handle actuator 144 in an initial position, such that a user can readily overcome the actuator detent mechanism by a slight motion of, or firm grasp upon, the handle actuator. In contrast, the actuator detent mechanism 452 may instead provide a “safing” function, in which the user is required to perform some affirmative action, such as releasing a latch or breaking a seal, before the handle actuator 144 can be moved axially and/or longitudinally with respect to the handle body 136.

Operation of the multi-lumen catheter assembly 100 is shown in the sequence of FIGS. 6A-6C. In FIG. 6A, the multi-lumen catheter assembly 100 is shown in a first condition, in which the distal inner catheter end 224 (shown in phantom line) is located longitudinally between the distal intermediate catheter end 116 and the distal outer catheter end 108. The handle actuator 144 is located adjacent the distal handle end 140 in this first position. The inner catheter member 220 is visible within the handle lumen 142 through an opening 658 in the handle body 136. An actuator stub 660 (shown in phantom line) extends from an inner surface (not shown) of the handle actuator 144 and is adapted to extend at least partially through the opening 658 from the handle actuator toward the longitudinal axis 104: In the first condition of FIG. 6A, the handle actuator 144 is in both a first actuator rotation position and a first actuator translation position. The handle actuator 144 may be selectively moved from the first actuator rotation position in an axial rotation direction (shown by arrow 654). The handle actuator 144 may also be selectively moved from the first actuator translation position in a longitudinal translation direction (shown by arrow 656) relative to the handle body 136.

In FIG. 6B, the multi-lumen catheter assembly 100 is shown in a second condition, in which the handle actuator 144 is longitudinally spaced from both the proximal and distal handle ends 138 and 140. As can be seen from the phantom-line depiction of the actuator stub 660, the handle actuator 144 has been axially rotated in the rotation direction 654 from the first actuator rotation position to a second actuator rotation position. The handle actuator 144 has also been longitudinally moved in the translation direction 656 from the first actuator translation position to a second actuator translation position.

A detent slot 662, extending substantially circumferentially around a portion of the handle body 136, is visible in FIG. 6B. The detent slot 662 interacts with the actuator stub 660 to prevent the handle actuator 144 from moving longitudinally relative to the handle body 136 when in the first actuator rotation position, and to permit the handle actuator 144 to move longitudinally relative to the handle body 136 when in the second actuator rotation position. One of ordinary skill in the art can readily design the detent slot 662 to have a size, shape, and position operative to interact with the actuator stub 660 in a desired manner. For example, at least a portion of the detent slot 662 could extend from the opening 658 at an acute angle (not shown) so that a user would be required to push the handle actuator 144 longitudinally opposite the translation direction 656 while simultaneously axially rotating the handle actuator in the rotation direction 654 to move the handle actuator from the first actuator rotation position to the second actuator rotation position.

Optionally, and regardless of the configuration or presence of the detent slot 662, the actuator detent mechanism 452 may be operative to selectively resist movement of the handle actuator 144 between the first and second actuator rotation positions.

In the transition from FIG. 6A to FIG. 6B, the handle actuator 144 is moved longitudinally from the first actuator translation position to the second actuator translation position. Due to the motion of the handle actuator 144, the distal intermediate catheter end 116 in the depicted arrangement is moved in the translation direction 656 to expose the distal inner catheter end 224, as shown in FIG. 6B. Since the proximal intermediate catheter end 114 is attached to the handle actuator 144, the intermediate catheter member 112 is pulled longitudinally through the handle lumen 142 by the handle actuator, also as shown in FIG. 6B.

From the second condition of FIG. 6B, the handle actuator 144 can be moved further in the translation direction 656, to place the multi-lumen catheter assembly 100 in the third condition, depicted in FIG. 6C. In FIG. 6C, the handle actuator 144 has moved longitudinally into a third actuator translation position. However, as can be seen by the position of the actuator stuff 660, the handle actuator 144 remains in the second actuator rotation position. Optionally, an additional detent slot (not shown) could be located at or near the proximal handle end 138 to allow the handle actuator 144 to be axially rotated back to the first actuator rotation position and thus selectively prevent the handle actuator 144 from being moved longitudinally from the third actuator translation position.

As can also be seen in FIG. 6C, the distal intermediate catheter end 116 is in a fully retracted position, located adjacent the distal outer catheter end 108, when the multi-lumen catheter assembly 100 is in the third condition. Throughout the sequence of FIGS. 6A-6C, the distal outer and inner catheter ends 108 and 224 have remained the same distance apart, with only the distal intermediate catheter end 116 changing position. The user could move the entire multi-lumen catheter assembly 100 to change the absolute positions of the distal outer and inner catheter ends 108 and 224. Regardless of absolute motion, however, the handle 134 is adapted to maintain the relative position of the outer and inner catheter members 102 and 220 while providing movement of the intermediate catheter member 112 relative thereto.

The actuator stub 660, protruding from the handle actuator 144, is depicted and described herein as interacting with the opening 658 in the handle body 136 to guide both rotational and translational movement of the handle actuator relative to the handle body. However, it is intuitively obvious that a protrusion (not shown) from the handle body 136 could instead interact with an opening (not shown) in the handle actuator 144 to provide either or both of the rotational and translational relative movements. Additionally, any other suitable structure(s), whether or not a protrusion/opening pair, could be used to guide the rotational and/or translational movement of the handle actuator 144 relative to the handle body 136.

Though the handle actuator 144 and other structures of the multi-lumen catheter assembly 100 are shown in certain positions (e.g., the first and second actuator rotation positions and the first, second, and third actuator translation positions) for ease of description, the so-described structures are not limited to these discrete positions and may be located in any number of intermediate positions between those depicted. Likewise, the described and depicted structures need not progress between the depicted positions in the order shown, but may be selectively moved from one position to another in any desired order and following any desired timing sequence. For example, the handle actuator 144 could start in the second actuator translation position, be moved to the first actuator translation position, and then pass through the second actuator translation position (and many other intermediate positions) en route to the third actuator translation position. Alternatively, a “one way” mechanism or structure could be provided to restrict the handle actuator 144 to a certain sequential motion, such as only from the first to second actuator rotation position and/or only from the first to second actuator translation position. Sequential restriction of this type could be especially useful when the multi-lumen catheter assembly 100 is used to release a compressed stent and reversal of the deployment operation could damage the previously released stent.

It is contemplated that the outer and inner catheter chucks 446 and 448 and the handle actuator 144 may each hold the respective ones of the outer, inner, and intermediate catheter members 102, 220, and 112 in any desired manner, through the use of means such as, but not limited to: chemically and/or physically adhesive materials; screws, nails, staples, or other penetrating fasteners; a mechanically interlocking/engaging structure, such as a threaded or barbed fit; a magnetically interlocking/engaging structure; a structure adapted for frictional engagement (an “interference fit”) or a compression fit; or any combination thereof.

It is also contemplated that the handle actuator 144 or another structure of the multi-lumen catheter assembly 100 may include a mechanical advantage device (not shown) operative to change the relative motion ratio between the handle actuator 144 and the intermediate catheter member 112 from the 1:1 relationship provided by the depicted multi-lumen catheter assembly to a greater or lesser ratio, thus providing desired movement of the intermediate catheter member relative to one or both of the outer and inner catheter members 102 and 220.

Finally, it is contemplated that the actuator detent mechanism 452 could include any desired mechanism or combination of reversible or unidirectional/one-time-only mechanisms adapted to prevent one or both of longitudinal translation and axial rotation of the handle actuator 144 with respect to the handle body 136. For example, chemical or physical adhesives, captured-ball, spring-pin, piezoelectric, cantilevered latch, magnetic, perforated structures, a lockout of an actuating motor, a compliant interference (e.g., a rubberized structure), a ratchet, a deflecting member (e.g., a flat spring), or any other suitable means may be used to provide the actuator detent mechanism 452.

While aspects of the present invention have been particularly shown and described with reference to the preferred embodiment above, it will be understood by those of ordinary skill in the art that various additional embodiments may be contemplated without departing from the spirit and scope of the present invention. For example, all structures of the multi-lumen catheter assembly 100 may be formed from any material, or combination of materials, as suitable to provide the finished multi-lumen catheter assembly with desired properties (e.g., the handle actuator 144 could be formed of or coated with a nonslip material for ease of grasping and operation by the user). The first, second, and third lumens 110, 118, and 226 need not extend completely between the proximal and distal ends of the outer, intermediate, and inner catheter members 102, 112, and 220, respectively. The handle 134 may be designed in an ergonomic manner, for ease of gripping and operation by the user. The outer, intermediate, and inner catheter members 102, 112, and 220 may each be of any desired lengths. One or more of the outer, intermediate, and inner catheter members could be omitted from the multi-lumen catheter assembly 100. The outer and inner catheter chucks 446 and 448 may be at least partially formed integrally with the handle 134 or any other structure of the multi-lumen catheter assembly 100, or may be provided separately therefrom. The handle lumen 142 could be smaller than depicted and/or the inner catheter member 220 could be larger than depicted, so that the inner catheter member substantially fills the handle lumen. The handle actuator 144 may be moved manually by the user, with or without an automatic assistance mechanism such as, but not limited to, a stepper motor, a pneumatic or hydraulic piston, a pulley system, or the like, or any combination thereof. The first, second, and third conditions of the multi-lumen catheter assembly 100 are defined and depicted as examples only, and are neither intended nor operative to restrict the order, direction, or sequence in which certain structures of the claimed invention interact. The detent slot 662 is depicted as extending from the opening 658, but may be separate from the opening. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. A handle for providing relative motion to a multi-lumen catheter assembly, the handle comprising:
an elongated handle body having a longitudinal axis, the handle body having longitudinally spaced proximal and distal handle ends;
an outer catheter chuck connected to the distal handle end and adapted to receive an outer catheter member;
an inner catheter chuck connected to the proximal handle end and adapted to receive an inner catheter member;
a handle actuator movably connected to the handle body, the handle actuator being adapted to receive an intermediate catheter member and to move the intermediate catheter member relative to the inner and outer catheter members, the intermediate catheter member being at least partially interposed between the inner and outer catheter members.

2. The handle of claim 1, wherein the inner catheter member has a third lumen and the handle comprises an inner catheter adapter connected to the inner catheter member and providing access to the third lumen, the inner catheter adapter being mounted on the handle body.

3. The handle of claim 1, being configured to maintain at least a portion of each of the outer, intermediate, and inner catheter members in a coaxial relationship with at least a portion of the other of the outer, inner, and intermediate catheter members.

4. The handle of claim 1, wherein the inner catheter member extends longitudinally through at least a portion of the handle body when received by the inner catheter chuck.

5. The handle of claim 4, wherein the handle body has a handle lumen, and the inner catheter member extends longitudinally through at least a portion of the handle lumen.

6. The handle of claim 1, wherein the outer catheter chuck includes retention means adapted to frictionally engage the outer catheter member.

7. The handle of claim 1, wherein the inner catheter chuck includes retention means adapted to frictionally engage the inner catheter member.

8. The handle of claim 1, wherein the handle actuator is slidably connected to the handle body and adapted for longitudinal motion relative to the handle body to move the intermediate catheter member relative to the inner and outer catheter members.

9. The handle of claim 1, wherein the handle actuator is axially rotatable between first and second actuator rotation positions, the handle actuator being prevented from moving longitudinally relative to the handle body in the first actuator rotation position, and the handle actuator being permitted to move longitudinally relative to the handle body in the second actuator rotation position.

10. The handle of claim 8, comprising an actuator detent mechanism operative to selectively resist movement of the handle actuator in the longitudinal direction.

11. The handle of claim 9, comprising an actuator detent mechanism operative to selectively resist rotation of the handle actuator in the axial direction.

12. A multi-lumen catheter assembly, comprising:
an outer catheter member having a longitudinal axis and longitudinally spaced proximal and distal outer catheter ends with a first lumen defined therebetween;
an intermediate catheter member having longitudinally spaced proximal and distal intermediate catheter ends with a second lumen defined therebetween, the intermediate catheter member being at least partially located within the first lumen;
an inner catheter member having longitudinally spaced proximal and distal inner catheter ends, the inner catheter member being at least partially located within the second lumen; and
a handle connected to the proximal outer catheter end, the proximal intermediate catheter end, and the proximal inner catheter end;
wherein the handle is adapted to maintain the relative position of the outer and inner catheter members while providing movement of the intermediate catheter member relative thereto.

13. The multi-lumen catheter assembly of claim 12, wherein the handle comprises:
a handle body having longitudinally spaced proximal and distal handle ends, the distal handle end affixed to the proximal outer catheter end and the proximal handle end affixed to the proximal inner catheter end;
a handle lumen extending longitudinally between the distal and proximal handle ends, the inner catheter member extending longitudinally through the handle lumen; and
a handle actuator affixed to the proximal intermediate catheter end and movably connected to the handle body;
wherein the handle actuator is selectively movable relative to the handle body to move the intermediate catheter member longitudinally relative to the outer and inner catheter members.

14. The multi-lumen catheter assembly of claim 13, wherein the inner catheter member has a third lumen defined between the proximal and distal inner catheter ends and the proximal handle end comprises an inner catheter adapter placing the proximal handle end in fluid communication with the third lumen.

15. The multi-lumen catheter assembly of claim 13, wherein the handle actuator is slidably connected to the handle body.

16. The multi-lumen catheter assembly of claim 13, wherein the handle actuator is selectively movable in a longitudinal direction relative to the handle body.

17. The multi-lumen catheter assembly of claim 13, wherein the handle actuator is axially rotatable between first and second actuator rotation positions, the handle actuator being prevented from moving longitudinally relative to the handle body in the first actuator rotation position, and the handle actuator being permitted to move longitudinally relative to the handle body in the second actuator rotation position.

18. The multi-lumen catheter assembly of claim 16, comprising an actuator detent mechanism operative to selectively resist movement of the handle actuator in the longitudinal direction.

19. The multi-lumen catheter assembly of claim 17, comprising an actuator detent mechanism operative to selectively resist movement of the handle actuator between the first and second actuator rotation positions.

20. The multi-lumen catheter assembly of claim 12, wherein the handle actuator is selectively movable relative to the handle body to move the distal intermediate catheter end longitudinally between the distal inner catheter end and the distal outer catheter end.

21. The multi-lumen catheter assembly of claim 12, wherein the handle actuator is selectively movable relative to the handle body to extend the distal intermediate catheter end longitudinally beyond the distal inner catheter end.

22. The multi-lumen catheter assembly of claim 12, wherein the outer, intermediate, and inner catheter members are at least partially arranged in a mutually coaxial relationship.

23. An intraluminal prosthesis delivery system comprising the multi-lumen catheter assembly of claim 12, wherein at least one of the distal outer catheter end, the distal intermediate catheter end, and the distal inner catheter end is operatively connectable to an intraluminal prosthesis cartridge.

24. A method of providing relative motion to a multi-lumen catheter assembly, the method comprising the steps of:
providing an outer catheter member having a longitudinal axis and longitudinally spaced proximal and distal outer catheter ends with a first lumen defined therebetween;
at least partially locating an intermediate catheter member within the first lumen, the intermediate catheter member having longitudinally spaced proximal and distal intermediate catheter ends with a second lumen defined therebetween;
at least partially locating an inner catheter member within the second lumen, the inner catheter member having longitudinally spaced proximal and distal inner catheter ends;
connecting a handle to the proximal outer catheter end, the proximal intermediate catheter end, and the proximal inner catheter end;
maintaining the relative position of the outer and inner catheter members; and
providing movement of the intermediate catheter member relative to the outer and inner catheter members.

25. The method of claim 24, comprising the steps of:
providing a handle body having longitudinally spaced proximal and distal handle ends and a handle lumen extending longitudinally therebetween;
affixing the distal handle end to the proximal outer catheter end;
affixing the proximal handle end to the proximal inner catheter end;
extending the inner catheter member longitudinally through the handle lumen;
affixing a handle actuator to the proximal intermediate catheter end;
movably connecting the handle actuator to the handle body; and
moving the intermediate catheter member longitudinally relative to the outer and inner catheter members by selectively-moving the handle actuator relative to the handle body.

26. The method of claim 25, wherein the step of movably connecting the handle actuator to the handle body comprises the step of slidably connecting the handle actuator to the handle body.

27. The method of claim 25, wherein the step of moving the intermediate catheter member longitudinally relative to the outer and inner catheter members by selectively moving the handle actuator relative to the handle body comprises the step of selectively moving the handle actuator in a longitudinal direction relative to the handle body.

28. The method of claim 25, wherein the handle actuator is axially rotatable between first and second actuator rotation positions, the method comprising the steps of:
preventing the handle actuator from moving longitudinally relative to the handle body in the first actuator rotation position; and
permitting the handle actuator to move longitudinally relative to the handle body in the second actuator rotation position.

29. The method of claim 28, comprising the step of selectively resisting movement of the handle actuator between the first and second actuator rotation positions with an actuator detent mechanism.

30. The method of claim 24, comprising the step of selectively moving the handle actuator relative to the handle body to move the distal intermediate catheter end longitudinally between the distal inner catheter end and the distal outer catheter end.

31. The method of claim 24, comprising the step of extending the distal intermediate catheter end axially beyond the distal inner catheter end by selectively moving the handle actuator relative to the handle body.
